(19) United States
(12) Patent Application Publication
(10) Pub. No.: US 2012/0265157 A1
(43) Pub. Date: Oct. 18, 2012

(54) EYE DROP CONTAINER WITH ALIGNMENT COMPONENT

(75) Inventors: Nien-Te Liu, Kaohsiung City (TW); Sung-Wei Hsieh, Kaohsiung City (TW)

(73) Assignee: Shu-Te University, Kaohsiung City (TW)

(21) Appl. No.: 13/337,470

(22) Filed: Dec. 27, 2011

(30) Foreign Application Priority Data
Apr. 12, 2011 (TW) 100112597

(51) Int. Cl. A61F 9/00; A61M 35/00

(52) U.S. Cl. 604/302

(57) ABSTRACT

The present invention provides an eye drop container with an alignment component, including a body, a spout, and a reflective surface. The body has a top surface. The spout has a bottom integrated with the top surface and an opening opposite to the bottom. The reflective surface is disposed on the top surface of the body and has a central hole, wherein the bottom of the spout is disposed in the central hole.

### BACKGROUND OF THE INVENTION

1. Field of the Invention

The present invention relates generally to an eye drop container, and more particularly, to an eye drop container with an alignment component having a reflective surface that can make the eye drops fall into the eye accurately.

2. The Prior Arts

An eye drop container is mainly filled with eye drops that is usually used to clear debris from the eye. Before a conventional eye drop container is used to make the eye drops fall into the eye, a spout of the eye drop container firstly needs to be toward the eye and be aligned with the eye, and then to make the eye drops fall into the eye. However, accurate alignment of the center of the eye with the spout of the conventional eye drop container is often hard, and it results in misses and wasted the eye drops during the conventional eye drop container is used to make the eye drops fall into the eye.

In view of above disadvantages in the arts, it is necessary to provide an eye drop container that can make the eye drops fall into the eye accurately.

### SUMMARY OF THE INVENTION

A primary objective of the present invention is to provide an eye drop container with an alignment component to hopefully achieve the objective that allows accurate placement of the eye drops in the eye.

In order to achieve the object, the present invention provides an eye drop container with an alignment component, comprising:

a body having a top surface;
a spout having a bottom integrated with the top surface and an opening opposite to the bottom; and
a reflective surface disposed on the top surface of the body and having a central hole,
wherein the bottom of the spout is disposed in the central hole.

Using the reflective surface in the present invention, the user can see the position of the opening of the spout corresponding to the eye clearly, and then the user can move the opening of the spout to the position above the center of the eye to achieve the object that allows accurate placement of the eye drops in the eye.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic drawing showing a structure of an eye drop container with alignment component according to the present invention.

### DETAILED DESCRIPTION OF THE PREFERRED EMBODIMENT

The accompanying drawing is included to provide a further understanding of the invention, and is incorporated in and constitute a part of this specification. The drawing illustrates embodiments of the invention and, together with the description, serve to explain the principles of the invention.

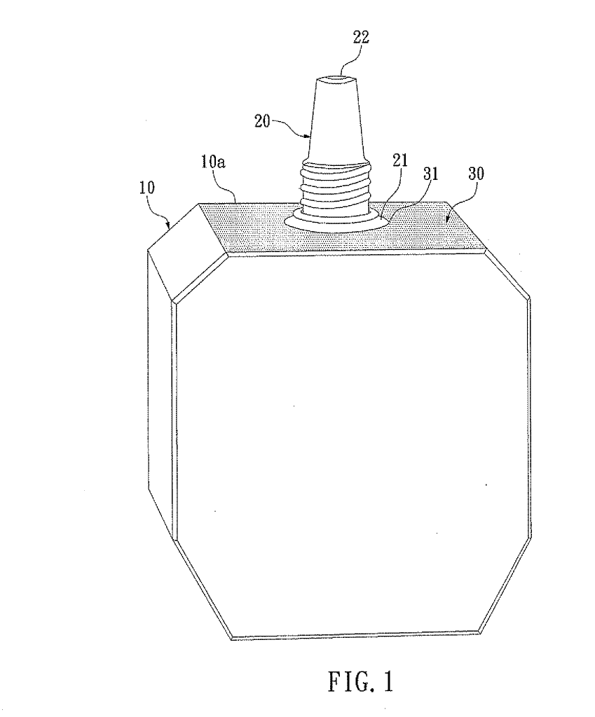

FIG. 1 is a schematic drawing showing a structure of an eye drop container with an alignment component according to the present invention. Please referring to FIG. 1, which shows a preferred embodiment of the present invention. An eye drop container with an alignment component includes a body 10, a spout 20, and a reflective surface 30. In this embodiment, the body 10 is used for filling with the eye drops, and it has a top surface 10a. The spout 20 has a bottom 21 integrated with the top surface 10a and an opening 22 opposite to the bottom 21. In this embodiment, the eye drops are discharged from the opening 22 of the spout 20. In addition, the reflective surface 30 is disposed on the top surface 10a of the body 10, and it has a central hole 31. In this embodiment, the bottom 21 of the spout 20 is disposed in the central hole 31 of the reflective surface 30. Besides, the reflective surface 30 can be a reflecting metal plate fixed to the top surface 10a of the body 10 in this embodiment, can be a reflecting metal film formed on the top surface 10a of the body 10 by using a film coating method in another embodiment, or can be a reflecting adhesive tape attached to the top surface 10a of the body 10 in yet another embodiment.

Using the reflective surface 30 in the present invention, the user can see the position of the opening 22 of the spout 20 corresponding to the eye clearly, and then the user can move the opening 22 of the spout 20 to the position above the center of the eye to achieve the effect that allows accurate placement of the eye drops in the eye.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. An eye drop container with an alignment component, comprising:
a body having a top surface;
a spout having a bottom integrated with the top surface and an opening opposite to the bottom; and
a reflective surface disposed on the top surface of the body and having a central hole,
wherein the bottom of the spout is disposed in the central hole.

2. The eye drop container with the alignment component of claim 1, wherein the reflective surface is a reflecting metal plate fixed to the top surface of the body.

3. The eye drop container with the alignment component of claim 1, wherein the reflective surface is a reflecting metal film formed on the top surface of the body by using a film coating method.

4. The eye drop container with the alignment component of claim 1, wherein the reflective surface is a reflecting adhesive tape attached to the top surface of the body.
